(19) United States
(12) Patent Application Publication
(10) Pub. No.: US 2012/0029655 A1
(43) Pub. Date: Feb. 2, 2012

(54) IMPLANTABLE XENOGRAFT PREPARED FROM A NON-HUMAN TISSUE PORTION

(75) Inventors: Guhathakurta Soma, Chennai (IN); Mammen Cherian Kotturathu, Chennai (IN)

(73) Assignee: INTERNATIONAL CENTRE FOR CARDIO THORACIC AND VASCULAR DISEASES, Chennai (IN)

(21) Appl. No.: 13/263,111

(22) PCT Filed: Jun. 10, 2009
(86) PCT No.: PCT/IN09/00335

§ 371 Date: Oct. 6, 2011

(30) Foreign Application Priority Data
Apr. 8, 2009 (IN) 818/CHE/2009

(51) Int. Cl. A61F 2/02

(52) U.S. Cl. 623/23.72

(57) ABSTRACT

An implantable xenograft intended for human recipients for correcting cardiovascular abnormalities. The xenografts are prepared from a non-human native tissue portion and are completely sterilized, acellular and exhibit a tensile strength generally higher than that of the native tissue. The uniqueness of the xenografts is that the outer surface of the tissue portion is impregnated with collagen nano-fibers. The implantable xenograft exhibits excellent storage stability, non-immunogenicity, non-thrombogenicity, non-cytotoxicity and non-calcifying properties.

### PRIORITY CLAIM

This patent application is a U.S. National Phase of International Patent Application No. PCT/IN2009/000335, filed Jun. 10, 2009, which claims priority to Indian Patent Application No. 818/CHE/2009, filed Apr. 8, 2009, the disclosures of which are incorporated herein by reference in their entirety.

### FIELD OF INVENTION

The present invention relates to implantable xenografts, processed from a non-human tissue portion. The present invention more specifically relates to the cardio vascular xenografts for correcting cardiovascular abnormalities.

### BACKGROUND

Xenograft is a graft of tissue taken from a donor of one species and grafted into a recipient of another species.

Implantation of xenografts finds wider applications in orthopedic, dental, cosmetic and cardiovascular surgeries. Since the awareness of diagnosing the heart diseases has increased and the modality of complex congenital heart diseases management has a wider spectrum the necessity for the implantable devices from xenogenic tissue manipulation has surfaced. This would be the life saving tools for children as well as adults.

Homograft valves are donated by patients and harvested after the patient dies. Generally such valves are not available during emergency situations or even otherwise not freely available due to various ethical reasons. Hence, tissue heart valves are usually made from animal tissues, either animal heart valve tissue or animal pericardial tissue.

The xenografts after implantation in individuals stimulate hyperacute rejection (HAR), soon after implantation. The rejection is surmounted by treating the xenografts by numerous techniques e.g. treatment of xenograft with chemical agents prior to implantation.

In the case of synthetic heart valves, it enables one to modify advantageously the properties of the heart valves by altering the monomers and/or the reaction conditions of the synthetic polymers. However U.S. Pat. No. 4,755,593 describes that synthetic heart valves are greatly associated with thromboembolism and mechanical failure. Although many synthetic and tissue based materials are used for forming heart valve replacements, they exhibit many disadvantages.

Allograft or xeno-grafts have important clinical advantages over non-biological synthetic biomaterials. Since the logistics of allograft availability is unpredictable, dependence on xeno-grafts as implants during surgeries has exponentially grown over the years.

U.S. Pat. No. 4,755,593 describes about tissue based heart valves. Although the valves demonstrate superior blood contacting properties relative to their synthetic counterparts, they are encountered with inferior in vivo stability.

Pericardial xenograft tissue valves have been introduced as alternatives to the synthetic and the tissue based valves (Ionescu, M. I. et al., Heart Valve Replacement With The Ionescu-Shiley Pericardial Xenograft, J. Thorac. Cardiovas. Surg. 73; 31-42 (1977)). But the pericardial xenograft tissue valves demonstrated calcification and durability problems (Morse, D, ed. Guide To Prosthetic Heart Valves, Springer-Verlag, New York, 225-232 (1985)).

Currently, available heart valve prostheses or conduits have major limitations which are mainly related to life long anticoagulation in mechanical valves and to degeneration in biological prostheses particularly in the young. Homo grafts from cadaver or from brain dead individual are the preferred valved conduit substitute in the reconstruction of the connection between right ventricle to the lung arteries in children with congenital heart disease. Limited durability, lack of growth and insufficient availability however remain problematic and that is why other options are surfacing. Among them synthetic implants, which are age old, are being used with all their disadvantages of thrombogenicity and difficult maneuverability due to lack of suitable conduit. Synthetic biodegradable grafts & hybrid of synthetic and biological tissue combination grafts are still in the developmental stages. Cardiovascular implants of biological tissue origin are the preferred one.

There is not even a single commercially available bioprosthetic heart valve which can mimic the native valve, can last long and can withstand the stresses of the native valve. Surgical replacement of the malfunctioning aortic valve with an allograft or another mechanical prosthetic valve is an alternative to bioprosthetic valve.

Heart valves made of xeno-grafts like porcine valve tissue or bovine pericardium have the advantage of low incidence of thrombogenicity without anti-coagulation. These valves have near normal functional accuracy like the native valves. However valve failure with structural dysfunction due to progressive tissue deterioration, including calcific degeneration and non-calcific damage are serious disadvantages, which undermine the attractiveness of such tissue valve substitutes. Though these xenografts are considered to have more physiological functions like native tissue, its early degeneration discourages its use. Hence, there is a long felt need in the field of tissue engineering for the development of an implantable xenograft which is non-immunogenic, non-thrombogenic, non-cytotoxic, non-calcifying and possessing good storage stability with a property of autologous cell deposition to become recipients own tissue (autograft).

The main goal of the current tissue engineering efforts is therefore the development of valve prosthesis or cardiovascular implants that combines unlimited durability with physiologic blood flow pattern mimicking normal native tissue and biologically non reactive surface properties. These will enable them to avoid life long anticoagulation and to be the suitable implants for the young ladies, small children and elderly people who particularly face the complications of anticoagulation.

All heart valve substitutes or available grafts lack the potential to grow and lack self repairing property. Hence strong efforts are directed towards the tissue engineering of heart valves as well as other cardiovascular implants, which would ideally behave as the natural human tissue after implantation.

Thus, there is a need for mechanically durable, flexible heart valves replacements which are capable of contacting the blood and are stable in vivo.

### SUMMARY

The primary object of the invention is to provide an implantable xenograft for a human recipient prepared from a non-human native tissue. The xenograft is processed to become acellular and has predetermined tensile strength.

Another object of the invention is to provide an implantable xenograft with excellent storage stability.

Yet another object of the present invention is to provide an implantable xenograft which is substantially non-immunogenic, non-thrombogenic, non-cytotoxic, non-calcifying and combinations thereof.

Further, object of the invention is to provide an implantable xenograft that allows the host tissue to grow over it and thereby gradually becoming an autograft comprising substantially of cell adhesion molecules such as collagen I, collagen IV, laminin and fibronectin.

### DESCRIPTION OF INVENTION

The present invention pertains to an implantable xenograft which finds application in cardiovascular surgeries. Xenograft tissue for clinical use has to cover many requirements, which would make it suitable for implantation in humans. It has been reported that calcific degeneration of the bioprosthetic material plays a major role in the failure of bioprosthetic and other biological substitutes. Calcium containing extra cellular fluid reacts with the membrane-associated phosphorus to yield calcium phosphate, which mineralizes on the cell membrane. Cellular residues, found in tissues treated with glutaraldehyde, primarily initiate tissue calcification.

Antigenicity due to the presence of native cells can be alleviated by decellularization, which is supposed to be a major factor for calcific degeneration and at the same time anticalcium treatment would reduce the degenerative changes as a whole. Prevention of blood protein seepage in the collagen matrix also plays a great role in preserving the life of the biological tissue.

An implantable xenograft is obtained by processing tissues preferably harvested from a group comprising of bovine jugular vein, bovine pericardium or porcine pulmonary artery. The harvested tissue is decellularised and made free of native cells and cell debris. The xenograft for implantation in to human recipient during cardiovascular surgeries is obtained by processing the harvested tissues by the following steps:

The first step involves treating the xenograft tissues with deoxycholic acid detergent in an antibiotic solution to disrupt all the cell membranes for initiating primary decellularisation of tissues to obtain partially decellularized tissues. Sterilization is automatically taken care by the use of antibiotic in this step.

The second step involves treating the said partially decellularized tissues with nucleases selected from the group consisting of DNAase, RNAase or a combination thereof, resulting in cell death to produce completely decellularized collagen tissue matrix and thereby preventing calcification of xenograft tissues upon implantation. The enzymes used in this step, also takes care of the sterilization.

The third step involves cross-linking of the completely decellularized collagen tissue matrix with heparin for preventing blood protein seepage. This cross-linking step involves attachment of heparin—within the receptors of collagen tissue matrix forming an artificial matrix protein complex on the decellularized collagen tissue matrix. Heparin treatment step also acts as a sterilizing step. The cross-linking step imparts strength and structural integrity to the xenograft to regulate the bio-remodelling of collagen by host cells when implanted.

The fourth step involves a specific intentional sterilization of the cross-linked decellularized collagen tissue matrix with formaldehyde.

The fifth step involves treatment of the sterilized decellularized collagen tissue matrix with heparin for repeated cross-linking and then subjecting to aldehyde capping using glutamic acid or lysine hydrochloride to eliminate the excess amount of residual aldehydes thereby effectively reducing detriment in the said matrix, inhibiting cellular toxicity and allowing autologous cell growth in the luminal intima and also creating subsequent layers.

The most novel step of offering a coating to the outer surface of the treated sterile decellularized collagen tissue matrix is done with collagen nano fibres, which thereby improve the strength of the treated sterile decellularized collagen tissue matrix enormously till it is repopularised with the recipients own cells.

The subsequent and the final step is preserving the nano collagen fiber coated sterile decellularized collagen tissue matrix in ethyl alcohol which is a non-toxic preservative.

In the above mentioned process the xenograft tissue undergoes multiple sterilization automatically with the reagents used for processing without incorporating a separate sterilizing agent. Besides this, a specific intentional sterilization using formaldehyde is also carried out.

Multiple sterilization of xenograft tissue effectively aids in killing or eliminating transmissible agents (such as fungi, bacteria, viruses, spore forms, etc.) from the surface of the collagen tissue matrix to yield a superior quality tissue.

Studies showed that sterilization of the decellularized tissue matrix with gamma-ray prior to implant causes considerable damage to the tissue. Hence, gamma ray sterilization is not practiced in this invention.

The deoxycholic acid is in the range of 0.5 to 1% in antibiotic and the tissue is treated with deoxycholic acid for a period 24 hours to 80 hours.

The decellularized tissue is then treated with nuclease for a period 8 hours to 15 hours, preferably treated with DNAase in the range 20 to 40 units to 100 ml of double distilled water; or treated with RNAase in the range 5 mg to 10 mg to 100 ml of double distilled water.

The enzyme-treated tissue is then sequentially processed with heparin in the range 30 to 70 units of heparin in 1 ml of balanced salt solution for initial cross-linking and formaldehyde in the range of 4% to 10% for sterilization followed by repeated cross linking.

The formaldehyde is preferably in the range 4% to 10%, and the duration of the sterilization is preferably for 8 hours to 15 hours.

The range of ethyl alcohol for preserving the processed tissue is 60% to 80%. The tissue is decellularized by treatments including anti-calcium treatment with 60-70% ethyl alcohol.

The decellularization process preferably comprises two steps, first step treating the tissues with 1% DCA detergent and subsequently treating the tissue with nucleases for enzymatic treatment.

The tissue process comprises two heparin treatments, the first heparin treatment prior to formalin sterilization and the second heparin treatment after the formalin sterilization.

### Processed Xenografts:

Each batch of processed grafts was subjected to stringent tests for mechanical and biocompatibility testing. Differential scanning calorimetry was done to prove the basic collagen stability and our tissues have acquired a very high level of stability by allowing heat flow between 89° C. to 118° C. (for normal collagen 80° C. is the standard).

Collagen conformity by Fourier Transform Infrared Spectroscopy has provided us with favourable results of basic normal collagen structure showing presence of amide, imide bond at the right wave numbers and the IR ratio remained around 1.

Tensile strength measurements of these tissues have been performed by Burst test on a universal testing machine and achieved the proper standard results. The implantable xenograft prepared from a non-human native tissue portion, is acellular and has a tensile strength is generally higher than that of the native tissue and is further characterized with the outer surface impregnated with collagen nano-fibers. The tissue is preserved for at least 9 months by exposing to a non-toxic chemical fixing agent. The processed xenografts have minimum tensile strength which is at least double the value of the native tissue. Biocompatibility tests results for immunogenicity and cytotoxicity are quite favourable and tissues are found to be non-cytotoxic and non immunogenic. Thrombo resistance property of the surface of the products in contact with human blood when checked also showed favorable results. All the above tests were conducted in FDA and CE approved laboratories for accreditation.

Finally the products are preserved in ethyl alcohol, which was considered to be an anticalcium agent too, apart from its anti-infective and preservative properties. Fourier transform infra red spectroscopy (FTIR) and differential scanning calorimetry (DSC) to assess the collagen property and stability in both the tissue groups have provided favourable results in the heparin group.

Lastly the animal experimentation had been done after informing the CPCSEA committee and after seeking the approval of animal research committee of the hospital. Twenty four sheep experiments had been performed to see the functionality and behavior of these products in different species. These are implanted subcutaneously in as well as in the abdominal cavity of the sheep and also as a cardiovascular implant in the circulatory system of sheep. None of the sheep died postoperatively due to implantation, they did not show any signs of local inflammation or systemic reaction, no haemolysis was detected and all of them were apyrexic through out. Explantation of those implants were checked with Von Kossa Stain to detect calcification and proved to be absent even after six months of implantation. The single layer collagen scaffold became tri-layered blood vessel in the sheep, when subjected to the sheep circulation by virtue of having cell adhesion molecule. The immunohistochemistry results proved the cell type depositions. Clinical observations have found that animals are all doing well. Only two bovine jugular veins which had been implanted as interposition grafts at the left internal jugular vein of the sheep were explanted and partial endothelialisation had been noticed in the lumen of the explanted BJV. But these two belonged to the group who hadn't heparin treatment and the sheep were not on any antiplatelet therapy. So far 13 sheep experiments were performed with group I (nonheparin treatment) & group II (heparin treated) tissues and all the results have gone in favour of heparin treated tissues. Explantation of those implants were checked with Von Kossa Stain to detect calcification and proved to be absent even after six months of implantation. The single layer collagen scaffold became tri-layered blood vessel in the sheep, when subjected to the sheep circulation by virtue of having cell adhesion molecule. The immunohistochemistry results proved the cell type depositions. Finally the products are preserved in ethyl alcohol, which was considered to be an anticalcium agent too, apart from its anti-infective and preservative properties. Fourier transform infra red spectroscopy (FTIR) and differential scanning calorimetry (DSC) to assess the collagen property and stability in both the tissue groups have provided favourable results in the heparin group.

Fluoroscopy reports confirm that these two explanted specimens did not show calcified opacification. No inflammation, aneurysm or dystrophic calcification was noted for the acelluar matrix vascular prosthesis and complete reendothelialization of the grafts in ovine has been reported. In a marked difference, this procedure of without heparin treatment found partial endothelialization of the acellular jugular vein lumen, which has provided the expectation of autologous cell seeding, which is the eventual goal in the heparin coated ones, i.e. the standardized processing. It is expected that post decellularization, heparin interaction along with anti-calcium measures by alcohol would prevent tissue degeneration and by tensile strength testing we have found out that shelf life could be easily 9 months as the tensile strength of the tissues remain 9 Mpascal even after 12 months. But for safety reason the shelf life we prescribe up to 6 months. In vitro and in vivo cytotoxicity test of the processed tissues were performed and they were found to be nontoxic.

Having successfully passed these stringent tests of biocompatibility and mechanical testing of these products and also proving them free of infection, clinical trial was done, After seeking permission from Institutional Ethical Committee, for no-option patients 371 bovine pericardial patches as cardiovascular patch material, and 78 bovine jugular veins, 82 porcine pulmonary artery conduit as cardiovascular conduits, when implanted on the right side of the heart showed great success in the clinical trials.

No calcification or thrombogenicity was detected so far in the human implants, no evidence of infective endocarditis or adverse reactions in patients during the trial test. Many small children and infants could be operated due to smaller sizes of the conduits which matched their vessels sizes. They never expressed presence of Gal epitope antigen in the recipient's blood signifying complete decellularisation.

Decellularisation of tissues using DCA and separate enzymatic digestions has produced clinically satisfactory results. Microscopically these tissues had shown satisfactory decellularization with intact tissue architecture. Subsequent processings to achieve longer life without calcific degeneration and probability of autologous cell seeding suggest these tissues will be the future answer for biological conduit or patches in clinical practice.

1. Implantable xenografts for human recipients prepared from a non-human native tissue portion, which is completely sterilized, which is acellular and has a tensile strength generally higher than that of the native tissue and is further characterized with the outer surface impregnated with collagen nano-fibers for further strength.

2. The implantable xenograft as claimed in claim 1 is prepared from native tissue portion selected from a group comprising of bovine jugular vein, bovine pericardium or porcine pulmonary artery.

3. The implantable xenograft as claimed in claim 1 is a prepared tissue which is a decellularised collagen tissue matrix.

4. The implantable xenograft as claimed in claim 1 is a prepared tissue which is a decellularised collagen tissue matrix cross-linked with heparin.

5. The implantable xenograft as claimed in claim 1 is characterized in the said prepared tissue has a minimum tensile strength which is at least double the value of tensile strength of the native tissue.

6. The implantable xenograft as claimed in claim 1 is characterized in the said prepared tissue has a minimum tensile strength for a period of at least nine months.

7. The implantable xenograft as claimed in claim 1, is characterized in the said collagen nano-fibers being a nano peptide.

8. The implantable xenograft as claimed in claim 1, is characterized in that the said prepared tissue is substantially non-immunogenic.

9. The implantable xenograft as claimed in claim 1, characterized in that the said prepared tissue is substantially non-thrombogenic.

10. The implantable xenograft as claimed in claim 1, is characterized in the said prepared tissue is substantially non-cytotoxic.

11. The implantable xenograft as claimed in claim 1, is characterized in the said prepared tissue is substantially non-calcifying.

12. The implantable xenograft as claimed in claim 1, is characterized in the said prepared tissue is substantially non-immunogenic or non-thrombogenic or non-cytotoxic or non-calcifying or combination thereof.

13. The implantable xenograft as claimed in claim 1 is characterized in that the said prepared tissue is an autograft comprising substantially of cell adhesion molecules such as collagen I, collagen IV, laminin, fibronectin and combination thereof.

14. The implantable xenograft as claimed in claim 1 is characterized in that the said prepared tissue is further exposed to a non-toxic preservative for preserving the said prepared tissue for at least 9 months.

15. The said non-toxic preservative of claim 14 is 60 to 80% ethyl alcohol.
